(19) United States
(12) Patent Application Publication
(10) Pub. No.: US 2023/0074814 A1
(43) Pub. Date: Mar. 9, 2023

(54) SYSTEM AND METHOD FOR PERSONALIZED OPTIMIZATION OF NUTRITION PLANS FOR USE DURING STAGES OF PRECONCEPTION, PREGNANCY, AND LACTATION/POST-PARTUM

(71) Applicant: Ali Mostashari, New York, NY (US)

(72) Inventors: Ali Mostashari, New York, NY (US); Raya Khanin, New York, NY (US); Klemo Vladimir, New York, NY (US); Mario Storga, New York, NY (US)

(73) Assignee: LifeNome Inc., New York, NY (US)

(21) Appl. No.: 17/902,974

(22) Filed: Sep. 5, 2022

Related U.S. Application Data
(60) Provisional application No. 63/241,852, filed on Sep. 8, 2021.

(51) Int. Cl. G16H 20/60; G06N 5/02

(52) U.S. Cl. CPC G16H 20/60; G06N 5/022

(57) ABSTRACT

A system for optimizing nutrition for women during stages of preconception, pregnancy, and lactation/postpartum is provided. The system comprises a computer and a calculator engine application executing on the computer that receives a request from a user device to create a dietary program for a woman. The application receives data describing the woman, the data including a current stage, the stage comprising one of preconception, pregnancy, and lactation/postpartum. The application compares the data to reference population data. The application also extracts material from a knowledge base describing at least one of levels of micro- and macro-nutrients at each stage and effects of various factors on levels of micro- and macro-nutrients at each pregnancy stage. The application also computes, via at least one algorithm, personalized micronutrients and macronutrients predisposition risk likelihoods and needs for the woman, the computation based at least on the received data, the comparison, and the extracted material.

### CROSS REFERENCE TO RELATED APPLICATIONS

The present non-provisional patent application is related to U.S. Provisional Patent Application No. 63/241,852 filed Sep. 8, 2021, the contents of which are incorporated herein in their entirety.

### FIELD OF THE DISCLOSURE

The present disclosure is in the field of pre-natal and post-natal health care for women. More particularly, the present disclosure provides systems and methods of computing micronutrients and macronutrients predisposition risk likelihoods and needs based at least on received data from a woman and developing a personalized dietary program for the woman based on the computed likelihoods and needs and other inputs including extensive data sources.

### BACKGROUND

Providing personalized nutrition advice during preconception, pregnancy, and lactation tailored to a woman's genetics and other variables is critical to ensure the health and wellness of women and babies. This is an issue of major importance for public health. Collecting large amounts of heterogeneous data from pregnant and lactating women can serve as a basis for improving nutrition recommendations and macro- and micro-nutrient guidelines by tailoring diets and recipes, tuning food recommendations for specific subgroups with dietary allergies, and/or dietary restrictions, and identifying at-risk groups for pregnancy complications.

Nutrition in pregnancy has been recognized for millennia as being important. The nutritional practices of many pregnant women often do not conform to well-known best practices. According to the American Dietetic Association, women of child-bearing age should maintain good nutrition through a lifestyle that optimizes maternal health and reduces the risk of birth defects, suboptimal fetal growth and development, and chronic health problems in their children.

Key components of a health-promoting lifestyle before and during pregnancy include appropriate weight gain, appropriate physical activity, consumption of a variety of foods, appropriate and timely vitamin and mineral supplementation, avoidance of alcohol, tobacco, and other harmful substances, and safe food handling. Pregnant women with excessive weight gain, hyperemesis, poor dietary patterns, phenylketonuria, certain chronic health problems, or a history of substance abuse should be referred to a registered dietitian for medical nutrition therapy.

Previous implementations have addressed personalized cooking-related information associated with preconception, pregnancy, birth, and postnatal care. Those subjects include personalized pregnancy, birth, postnatal care-related information providing service method, apparatus and system, and wellness service providing method and system utilizing mother and baby health data.

The prior art has provided nutrition-related information based on data from wearable biometric devices and health evaluation. Personalized information corresponding to the calculated pregnancy birth health index may include pregnancy birth control information, pregnancy and childbirth exercise information, pregnancy and childbirth nutrition information, pregnancy and childbirth education information, and pregnancy and delivery products and service information.

The prior art devoted to personalized nutrition in general, and specifically in personalized nutrition during preconception, pregnancy, lactation, and postnatal care, has inadequately addressed the following issues.

First, prior approaches do not take into consideration genetics data for women during preconception, pregnancy, and lactation stages. This data is needed for integration with other non-genetics information to calculate risks for complications and nutritional imbalances or inadequacies.

Second, nutrition programs generally identify nutrients that a woman is deficient in and then recommend foods or supplements that contain high amounts of these nutrients. But such programs disregard the contents of other, potentially unhealthy, nutrients in the context of pregnancy.

Cases when a woman has dietary restrictions during pregnancy and lactation are not properly handled. For example, gluten free diets have been shown to contain higher amounts of sugars and lower amounts of several essential nutrients such as vitamin B12. Early approaches do not provide nutrition optimization that takes into consideration multiple factors. Another example is iron or folate supplementation for women without taking into consideration the woman's diet and genetics.

Third, existing nutrition programs are not self-learning systems. The existing programs do not provide tools for collecting large amounts of heterogeneous data, analyze such data to obtain relevant nutrition insights, and offer newly learned nutrition recommendations to new consumers.

Shortcomings therefore exist regarding systems and methods and that offer personalized nutrition advice to pregnant and lactating women. The disclosed systems and methods address the shortcomings of previous approaches.

### BRIEF DESCRIPTION OF THE FIGURES

FIG. 1 is a block diagram of a system for optimizing nutrition for women during stages of preconception, pregnancy, and lactation/postpartum according to an embodiment of the present disclosure.

FIG. 2 is a block diagram of a system for optimizing nutrition for women during stages of preconception, pregnancy, and lactation/postpartum according to an embodiment of the present disclosure.

### DETAILED DESCRIPTION

Systems and methods provided herein optimize nutrition for women during stages of preconception, pregnancy, and lactation/postpartum. Based on information collected from the woman and other sources, systems and methods provided herein compute macro- and micronutrient needs and ranges for the woman. These computations may be performed based in part on the woman's stage, activity level, age, and medical history by utilizing machine learning methods.

Based on the computed needs and ranges, the system algorithmically generates a personalized dietary program including meal plans and shopping lists for the woman. The meal plans may be set for regular intervals such as daily, weekly, monthly, or trimester as well the woman's stage that may be trimester, pregnancy week, preconception, or post-partum. The system may complete this task by performing multi-variable optimization of food and recipe databases for the woman. These actions are based on the woman's calculated ranges for macro- and micronutrients, dietary restrictions, food allergies and sensitivities, and personal preferences.

The system also includes reporting and feedback processes that allow and encourage the woman to submit feedback about a dietary program they have been given. Additionally, feedback, when received from many dietary program recipients, may be used to make adjustments to some of the algorithms and models used by the system and some of the stored dietary programs.

A desired result of providing better dietary programs to many women may be achieved. The system effectively learns from feedback, both supportive and critical, to upgrade and fine tune dietary programs shown to be effective and overhaul and eliminate less successful programs.

The system infers linkages between diet and adverse events before, during and after pregnancy by utilizing the algorithms and drawing upon databases containing previous feedback and lessons learned. The system also identifies groups of women with similar risks for adverse effects arising from dietary programs. Adverse effects may include reactions to foods and recipes that cause or ease nausea, for example.

The data collected from the woman in many embodiments includes genetics data and non-genetics data. Genetics data comprise output from genotyping array or sequencing data. The data may be provided by the woman or third parties.

Non-genetics data includes one of preconception, pregnancy, or lactation/postpartum stage. Non-genetics data also includes age, height, pre-pregnancy weight, height, physical activity level, medical history, and physiological parameters. Non-genetics data further includes data related to pregnancy/postpartum/lactation, data including weight gain or loss, appetite, food cravings and aversions, morning sickness, and nausea, food preferences, food sensitivities, and allergies.

The system compares the genetics and non-genetics data to data or other material extracted from a reference population database that describes other women of similar age, medical history, and stage. The system also draws on other knowledge bases containing at least information about levels of micro- and macro-nutrients at each stage and effects of various factors on levels of micro- and macro-nutrients at each stage.

A calculator engine application or module processes the genetics and non-genetics data, results of comparisons with the reference population database, and knowledge base material. From those inputs, the calculator engine application determines micro- and macro-nutrient needs and ranges for the subject woman. These needs and ranges are further based on guidelines for pregnant and lactating women and account for the women's pregnancy/lactation stage, maternal age, pre-pregnancy body mass index (BMI), physical activity, rate of weight gain during pregnancy, and rate of weight loss postpartum/lactation.

A recommendation engine application or module receives the micro- and macro-nutrient needs and ranges from the calculator engine. The recommendation engine designs the dietary program for the subject woman. Using at least one algorithm, the recommendation engine application develops personalized dietary recommendations comprising foods, recipes, weekly shopping lists, and meal plans. The recommendations consider the woman's dietary restrictions, food allergies and sensitivities, and personal preferences. The recommendations may include personalized vitamin and mineral supplements.

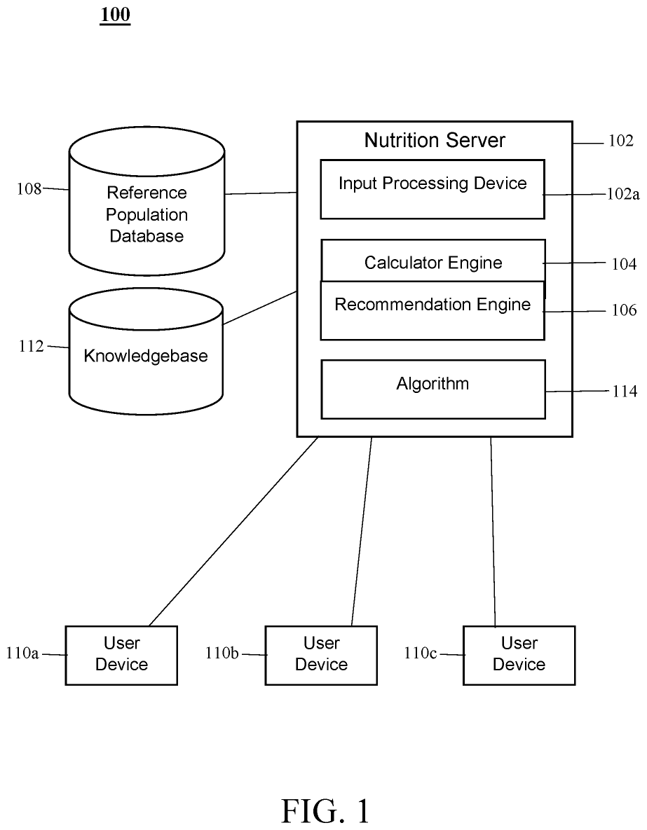

Turning to the figures, FIG. 1 is a block diagram of a system for optimizing nutrition for women during stages of preconception, pregnancy, and lactation/postpartum according to an embodiment of the present disclosure.

FIG. 1 illustrates components and interactions of a system 100 of receiving genetics and non-genetic data for a woman, calculating personalized calories, including macro- and micro-nutrient needs and ranges based on the data, and generating by a recommender engine application, personalized dietary recommendations.

The system 100 comprises a nutrition server 102, a calculator engine 104, a recommender engine 106, and a reference population database 108. The system 100 also comprises a plurality of user devices 110a-c in possession of women used by the women to submit material to the nutrition server 102 and to receive dietary programs and other material from the nutrition server 102 and other components. While quantity three user devices 110a-c are depicted in FIG. 1 and provided by the system 100, in embodiments more than or less than quantity three user devices 110a-c may be provided. The system 100 also comprises a knowledge base 112 and at least one algorithm 114.

The nutrition server 102 may be a single or multiple physical computers situated at one or multiple geographic locations. While the calculator engine 104 and the recommender engine 106 are depicted in FIG. 1 as contained by or components of the nutrition server 102 and executing on the nutrition server 102, in embodiments the calculator engine 104 and the recommender engine 106 may be separate components or software executing on separate devices proximate or remote from the nutrition server 102.

While referred to as engines, the calculator engine 104 and the recommender engine 106 may be combinations of hardware and software applications or entirely software applications Components described throughout herein as modules, submodules, or devices may be physical devices, combinations of a physical device and software, or entirely software.

The nutrition server 102 receives genetics and non-genetics data from one of the user devices 110a-c. The received data is processed by an input processing device 102a of the nutrition server 102 and stored in the reference population database 108. The received data is also provided to the calculator engine 104 to determine macro- and micro-nutrient needs and ranges for the woman.

Based on the macro- and micro-nutrient needs and ranges determined by the calculator engine 104, the recommender engine 106 generates dietary recommendations, foods, and recipes. Feedback regarding liking/disliking of dietary recommendations and adverse effects such as morning sickness, or nausea, may be provided back to the recommender engine 106, the calculator engine 104, and/or the reference population database 108 to improve future personalized dietary recommendations. The calculator engine 104 and the recommender engine 106 may use algorithm 114 to complete their tasks.

Additional data, such as the rate of weight gain during pregnancy, or weight loss postpartum, may be collected from the woman and submitted to the recommender engine 106, the calculator engine 104, or the reference population database 108 to improve future personalized dietary recommendations, and to discover relationships between genetics, dietary consumption, and preconception, pregnancy, lactation/postpartum traits.

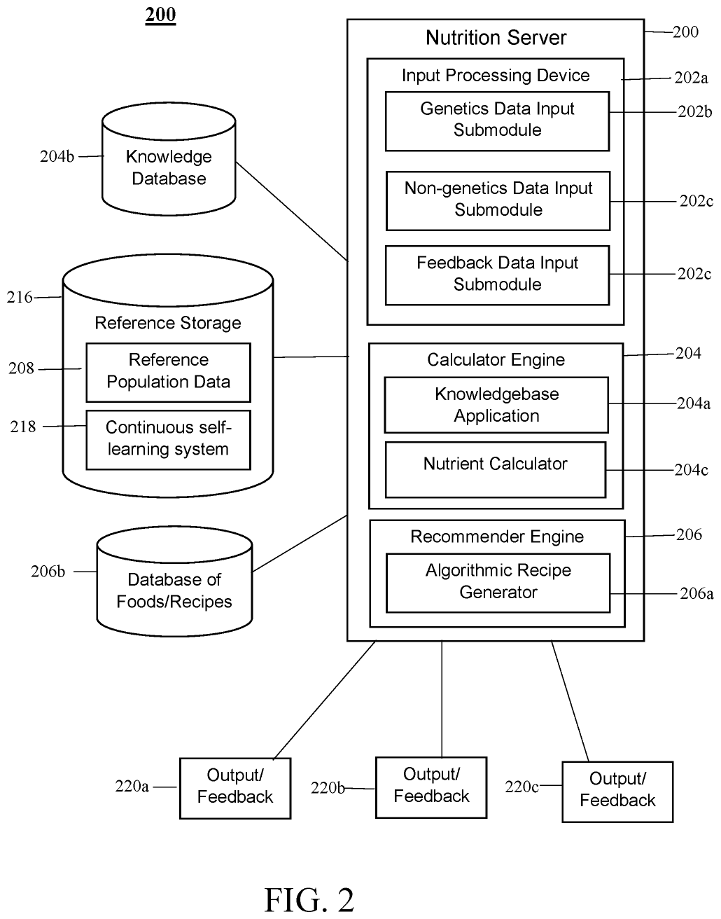

FIG. 2 is a block diagram depicting an embodiment of a system 200 provided herein. Components of the system 200 are indexed components of the system 100. A nutrition server 202 is provided. An input processing device 202a, reference population database 208, calculator engine 204, and recommender engine 206 are provided by the system 200 as in the system 100.

The input processing device 202a includes three submodules or applications comprising a genetics data input submodule 202b, a non-genetics data input submodule 202c, and a feedback data input submodule 202d. In various embodiments, data input is done via a web, or mobile application at home, or in an outpatient clinical environment.

The nutrition server 202 receives genomics data from various sources via genetics data input submodule 202b that may be integrated with external information providers. In some embodiments, input data may be a file with genotype data uploaded by an individual, by external genotyping or sequencing service/company using a generic or proprietary application programming interface (API), or by a third party, for example, physicians, dieticians, and aestheticians. In other embodiments, input data is a file with RNA expression data or protein abundance data. This data may be uploaded by an individual, by an external sequencing service/company using generic or proprietary API, or by a third party, for example, physicians, dieticians, and aestheticians. Genomics data is pre-processed and analyzed using bioinformatics methods directed to obtaining quantifiable results to enable further assessments.

The nutrition server 202 receives non-genetics data from various sources via the non-genetics data input submodule 202c. Non-genetics data may include data about a woman's age, ethnicity, preconception/pregnancy/postpartum stage, demographics, height, weight, activity level, diet, habits, lifestyle, medical history, geolocation, environment, and preferences. Non-genetics data may contain data from physiological tests, for example, blood, urine, and stool, data from wearables, sensors, imaging data from professional devices or smartphones, and other relevant devices.

The non-genetics data input submodule 202c, which may be partially integrated with external information providers, enables input of non-genetics information by generic or proprietary API from imaging devices, sensors, wearables, and other relevant devices, or third-party expert reports, for example physicians, dieticians, and aestheticians. The non-genetics data input submodule 202c submodule also enables self-reported questionnaires or data input by third parties.

The feedback data input submodule 202d is utilized when the woman provides reviews, survey responses, or other feedback to the system 200 about a dietary program provided to the woman. The feedback data input submodule 202d receives feedback from the woman about specific recipes and food recommendations and likes/dislikes. The feedback data input submodule 202d may also receive reports of adverse effects such as morning sickness, nausea, weight gain during pregnancy or weight loss postpartum, blood pressure, pregnancy complications, baby gestational age, baby weight, and lactation issues.

Upon receipt of genetic and non-genetic data, input processing device 202a shares the received data with a reference population database 208 which is a repository of genetic and non-genetic data for a large number of expectant women and postpartum women as well as women in preconception. The reference population database 208 is a component of reference storage 206. The data stored in the reference population database 208 is continuously updated with new entries received from women at various stages. The reference population database 208 can also be updated by bulk downloads of genetics data from multiple women as well as non-genetic data from third parties, and databases and other repositories not directly associated with the system 200.

Feedback data, received from an expectant or postpartum woman or from other parties, is also propagated to the reference population database 208, and, after processing with self-learning system 218, may also be further transmitted to the calculator engine 204 and recommender engine application 206 to improve assessment of needs and future personalized dietary recommendations, foods, and recipes.

A continuous self-learning system 218 may thereby be set into place. For example, by analyzing via computational algorithms and collected data, the system may infer that women with specific genetic variations are more likely to have more morning sickness in the first trimester if they consume specific foods. Similarly, the system may learn that specific foods and recipes help women deal with morning sickness and nausea.

The reference storage 216 is an information source for computing personalized macro- and micro-nutrient predisposition risk likelihoods and needs performed by the calculator engine application 204. As with the reference population database 108 of the system 100, the reference population data 208 stored in reference storage 216 and provided by the system 200 may be a single database or multiple databases situated at a single or multiple geographic locations.

The calculator engine 204 comprises a knowledge base application 204a that organizes and dynamically structures state-of-the art knowledge in a knowledge database 204b related to macro and micronutrients effect on the various stages of preconception, pregnancy, and postpartum/lactation. The knowledge database 204b contains material on at least:
(i) levels of micro- and macronutrients at different stages of preconception, pregnancy, and lactation/postpartum as recommended, by ob-gyns and nutritionists;
(ii) effects of medical history, age, biometric data, lifestyle factors, dietary restrictions on levels of micro and macronutrients at different stages of preconception, pregnancy, and lactation/postpartum; and
(iii) effects of genetic variations on levels of micro and macronutrients at different stages of preconception, pregnancy, and lactation/postpartum.

The calculator engine 204 receives genetics and non-genetics data from the input processing device 202a. The calculator engine 204 compares the individual genetics and non-genetics data to stored material in the reference population data 208. The calculator engine 204 computes personalized macro- and micro-nutrient predisposition risk likelihoods and needs based on the received data and knowledge base 204b material. The calculator engine 204 may perform these computations of likelihoods and needs using at least one algorithm that may be proprietary and/or developed by a third-party source. The calculator engine 204 also includes a nutrient calculator 204c.

The computed macro- and micro-nutrient needs and ranges for the individual woman are transmitted to the recommender engine 206 to generate dietary recommendations, foods, and recipes. The input processing device 202a may transmit additional data collected from the woman to the recommender engine application 206.

In some embodiments, additional data collected from the woman may comprise dietary restrictions, preferences, allergies, and sensitivities. The recommender engine 206 algorithmically generates dietary recommendations, foods, recipes, daily and weekly meal planners, food shopping lists, and dietary tips. These actions may be based on the calculated macro- and micro-nutrient needs and additional data transmitted from the input processing device 202a.

The recommender engine 206 has access to a database of foods and recipes 206b. By performing multi-variable optimization, the recommender engine 206 generates weekly shopping lists and meal plans for a woman based on the woman's ranges for macro- and micro-nutrients, dietary restrictions, food allergies and sensitivities, and personal preferences. The recommender engine 206 relies upon an algorithmic recipe generator 206a to assist in creating recipes.

The database of foods and recipes 206b may have contents contributed, via feedback, by women who have been using the system 200. In embodiments, the recipes and foods are curated, via feedback, by women who have been using the system 200, some long after giving birth or otherwise completing or experiencing termination of pregnancy. For example, a specific recipe may be upvoted for women who have morning sickness during the first trimester of pregnancy. The recommender engine 206 and other components have access to a reference storage 216 which hosts the reference population data 208 and the self-learning system 218 that may be an ML discovery module.

Feedback provided by expectant women, postpartum women, and others may be done via a submodule that collects responses from provided dietary recommendations. Specifically, the feedback data may comprise liking/disliking dietary recommendations, foods, and recipes. Feedback data related to dietary recommendations, foods, and recipes are stored in a database of foods/recipes 206b. The feedback data may then be transmitted to the reference population data 208, and, after processing, be further transmitted to the calculator engine 204 and to the recommender engine 206 to improve future personalized dietary recommendations, foods, and recipes. As noted, a continuous self-learning system 218 may thereby be set in place.

The system 200 also comprises components depicted and enumerated as output/feedback 220a-c which may be equivalent or similar to the user devices 110a-c provided by the system 100 and depicted in FIG. 1. Output/feedback 220a-c receive dietary programs and provide feedback to the nutrition server 202. While quantity three output/feedback 220a-c are depicted in FIG. 2 and provided by the system 200, in embodiments more than or less than quantity three output/feedback 220a-c may be provided.

In an embodiment, a system for optimizing nutrition for women during stages of preconception, pregnancy, and lactation/postpartum is provided. The system comprises a computer and a calculator engine application executing on the computer that receives a request from a user device to create a dietary program for a woman. The application also receives data describing the woman, the data including a current stage, the stage comprising one of preconception, pregnancy, and lactation/postpartum. The application also compares the data to reference population data. The application also extracts material from a knowledge base describing at least one of levels of micro- and macro-nutrients at each stage and effects of various factors on levels of micro- and macro-nutrients at each pregnancy stage. The application also computes, via at least one algorithm, personalized micronutrients and macronutrients predisposition risk likelihoods and needs for the woman, the computation based at least on the received data, the comparison, and the extracted material.

The calculator engine sends the computed personalized micronutrients and macronutrients predisposition risk likelihoods and needs to a recommender engine application for development of a dietary program. The data describing the woman further comprises at least one of genetics data, non-genetics data, and longitudinal pregnancy progress and lactation data.

The genetics data comprises at least one of DNA data, obtained via genotyping array or sequencing, RNA expression data, protein abundances, methylation data. The non-genetics data comprises at least one of age, height, weight, ethnicity, medical history, diet, demographics, and stage, wherein stage comprises one of preconception, pregnancy, and postpartum.

The material from the knowledge base further comprises material describing levels of micronutrients and macronutrients at each stage as recommended by physicians and nutritionists. The material from the knowledge base further comprises material describing effects of medical history, age, biometric data, lifestyle factors, and dietary restrictions on levels of micronutrients and macronutrients at each stage. The material from the knowledge base further comprises material describing effects of genetic variations on levels of levels of micronutrients and macronutrients at each stage.

The application further performs the computation by using machine learning methods. The application further incorporates responses to questionnaire responses provided by the woman.

In another embodiment, a system for optimizing nutrition for women during stages of preconception, pregnancy, and lactation/postpartum is provided. The system comprises a computer and a recommender engine application executing on the computer that receives personalized micronutrients and macronutrients predisposition risk likelihoods and needs for a woman. The system also receives additional data associated with the woman comprising at least one of dietary restrictions, preferences, allergies, and sensitivities. The system also receives a request to generation a dietary program for the woman. The system also algorithmically generates, based at least on the risk likelihoods, and needs and on the additional data, the requested dietary program comprising at least one of dietary recommendations, recipes, daily and weekly meal planners, and shopping lists for the woman.

The recommender engine application receives the personalized micronutrients and macronutrients predisposition risk likelihoods and needs from a calculator engine application. The personalized micronutrients and macronutrients predisposition risk likelihoods and needs received from the calculator engine application are based on at least one of genetic and non-genetic information describing the woman.

Generation of the dietary recommendations further comprises personalized vitamin and mineral supplements. The recommender application makes adjustments to dietary programs based on feedback received from women on previously provided programs. The recommender engine in developing the requested dietary program further draws upon stored data describing previous dietary program results including adverse reactions.

In yet another embodiment, a method for optimizing nutrition for women during stages of preconception, pregnancy, and lactation/postpartum. The method comprises a computer receiving feedback from at least one user device to a dietary program, the program previously provided to at least one woman. The method also comprises the computer adding the feedback to a database containing previous feedback. The method also comprises the computer applying at least one algorithm to the received feedback and to the previous feedback. The method also comprises the computer inferring, based on results generated by the applied algorithm, at least relationships between adverse effects during pregnancy and diet. The method also comprises the computer identifying, based at least on the inferred at least relationships, groups of women with similar risks for the adverse effects.

The feedback comprises reports of liking/disliking dietary recommendations, foods, and recipes provided in the dietary program and adverse effects of the dietary program. The adverse effects comprise at least one of allergic reactions, nausea, vomiting, and one of unexpected weight loss and weight gain.

The computer subjects the inferred at least relationships between adverse effects during pregnancy and diet to further analysis and selectively provided to a reference population database. The computer performing the analysis based at least on stages of women, the stages comprising preconception, pregnancy, and lactation/postpartum. The computer including in the analysis correlating of adverse effects with genetic and non-genetic data provided by women experiencing the effects.

What is claimed is:

1. A system for optimizing nutrition for women during stages of preconception, pregnancy, and lactation/postpartum, comprising:
a computer; and
a calculator engine application executing on the computer that:
receives a request from a user device to create a dietary program for a woman,
receives data describing the woman, the data including a current stage, the stage comprising one of preconception, pregnancy, and lactation/postpartum,
compares the data to reference population data,
extracts material from a knowledge base describing at least one of levels of micro- and macro-nutrients at each stage and effects of various factors on levels of micro- and macro-nutrients at each pregnancy stage, and
computes, via at least one algorithm, personalized micronutrients and macronutrients predisposition risk likelihoods and needs for the woman, the computation based at least on the received data, the comparison, and the extracted material.

2. The system of claim 1, wherein the calculator engine sends the computed personalized micronutrients and macronutrients predisposition risk likelihoods and needs to a recommender engine application for development of a dietary program.

3. The system of claim 1, wherein the data describing the woman further comprises at least one of genetics data, non-genetics data, and longitudinal pregnancy progress and lactation data.

4. The system of claim 3, wherein the genetics data comprises at least one of DNA data, obtained via genotyping array or sequencing, RNA expression data, protein abundances, methylation data.

5. The system of claim 3, wherein the non-genetics data comprises at least one of age, height, weight, ethnicity, medical history, diet, demographics, and stage, wherein stage comprises one of preconception, pregnancy, and postpartum.

6. The system of claim 1, wherein the material from the knowledge base further comprises:
material describing levels of micronutrients and macronutrients at each stage as recommended by physicians and nutritionists,
material describing effects of medical history, age, biometric data, lifestyle factors, and dietary restrictions on levels of micronutrients and macronutrients at each stage, and
material describing effects of genetic variations on levels of levels of micronutrients and macronutrients at each stage.

7. The system of claim 1, wherein the application further performs the computation by using machine learning methods.

8. The system of claim 1, wherein the application further incorporates responses to questionnaire responses provided by the woman.

9. A system for optimizing nutrition for women during stages of preconception, pregnancy, and lactation/postpartum, comprising:
a computer; and
a recommender engine application executing on the computer that:
receives personalized micronutrients and macronutrients predisposition risk likelihoods and needs for a woman,
receives additional data associated with the woman comprising at least one of dietary restrictions, preferences, allergies, and sensitivities,
receives a request to generation a dietary program for the woman, and
algorithmically generates, based at least on the risk likelihoods and needs and on the additional data, the requested dietary program comprising at least one of dietary recommendations, recipes, daily and weekly meal planners, and shopping lists for the woman.

10. The system of claim 9, wherein the recommender engine application receives the personalized micronutrients and macronutrients predisposition risk likelihoods and needs from a calculator engine application.

11. The system of claim 10, wherein the personalized micronutrients and macronutrients predisposition risk likelihoods and needs received from the calculator engine application are based on at least one of genetic and non-genetic information describing the woman.

12. The system of claim 9, wherein generation of the dietary recommendations further comprises personalized vitamin and mineral supplements

13. The system of claim 9, wherein the recommender application makes adjustments to dietary programs based on feedback received from women on previously provided programs.

14. The system of claim 9, wherein the recommender engine in developing the requested dietary program further draws upon stored data describing previous dietary program results including adverse reactions.

15. A method for optimizing nutrition for women during stages of preconception, pregnancy, and lactation/postpartum, comprising:
a computer receiving feedback from at least one user device to a dietary program, the program previously provided to at least one woman;
the computer adding the feedback to a database containing previous feedback;
the computer applying at least one algorithm to the received feedback and to the previous feedback;
the computer inferring, based on results generated by the applied algorithm, at least relationships between adverse effects during pregnancy and diet; and
the computer identifying, based at least on the inferred at least relationships, groups of women with similar risks for the adverse effects.

16. The method of claim 15, wherein the feedback comprises reports of liking/disliking dietary recommendations, foods, and recipes provided in the dietary program and adverse effects of the dietary program.

17. The method of claim 15, wherein the adverse effects comprise at least one of allergic reactions, nausea, vomiting, and one of unexpected weight loss and weight gain.

18. The method of claim 15, further comprising the computer subjecting the inferred at least relationships between adverse effects during pregnancy and diet to further analysis and selectively provided to a reference population database

19. The method of claim 18, further comprising the computer performing the analysis based at least on stages of women, the stages comprising preconception, pregnancy, and lactation/postpartum.

20. The method of claim 18, further comprising the computer including in the analysis correlating of adverse effects with genetic and non-genetic data provided by women experiencing the effects.
